(19) United States
(12) Patent Application Publication
(10) Pub. No.: US 2014/0243296 A1
(43) Pub. Date: Aug. 28, 2014

(54) Organic Compounds

(71) Applicant: Jagotec AG, Muttenz (CH)

(72) Inventors: Pascal Grenier, Kappelen (FR); Guy Vergnault, Kembes (FR); Patricia Ibarra, Solothurn (CH)

(73) Assignee: JAGOTEC AG, Muttenz (CH)

(21) Appl. No.: 14/352,937

(22) PCT Filed: Oct. 5, 2012
(86) PCT No.: PCT/EP2012/069793

§ 371 Date: Apr. 18, 2014

(30) Foreign Application Priority Data
Oct. 21, 2011 (GB) 1118182.3

(51) Int. Cl. A61K 9/24; A61K 31/635; A61K 9/20; A61K 31/427

(52) U.S. Cl. CPC A61K 9/209; A61K 31/427; A61K 31/635; A61K 9/2013
USPC 514/158; 548/204; 514/365; 549/464

(57) ABSTRACT

A treatment regimen and dosage form comprising ritonavir and darunavir and their use in the treatment of HIV infection.

The present invention is concerned with methods and compositions for use in the treatment of HIV infection, and in particular such compositions and methods for improving the efficacy of HIV protease inhibitors.

Infection by HIV continues to be a serious human health problem. Methods of treating HIV include the administration of agents that inhibit the activity of viral enzymes and interfere with the life-cycle of the virus.

HIV protease inhibitors are known in the art to be useful in the treatment of HIV infection. The protease inhibitors include the compounds saquinavir, ritonavir, indinavir, nelfmavir, amprenavir, lopinavir, atazanavir, fosamprenavir, tipranavir and darunavir.

The protease inhibitors are potent anti-viral agents that when used in combination have dramatically changed the history of the HIV disease. However, the use of HIV protease inhibitors is limited somewhat by a range of factors, principally the poor oral bioavailability, which diminishes the convenience of administration of these drugs.

The cytochrome P 450 (CYP) family of enzymes (particularly the 3A4 isoform) can provide a significant barrier to uptake of the protease inhibitors. They are present at various sites throughout the body including the gut lumen and liver. The human gut provides the first site for CYP metabolism of orally delivered drugs; whereas CYP in the human liver provides a second site for CYP metabolism.

Protease inhibitors have been found to interact with CYP, particularly CYP 3A4 isoform. However, the affinity of the protease inhibitors towards the enzyme differs markedly and ranges from weak inhibition (saquinavir and amprenavir are weak inhibitors) to modest inhibition (nelfinavir and indinavir) to potent inhibition (ritonavir).

An additional barrier to drug uptake is P-glycoprotein (Pgp) found in the gut wall. Once again, the protease inhibitors demonstrate widely varying degrees of affinity for Pgp. Indinavir exhibits substantially no inhibitory effect whereas ritonavir has a potent effect.

Given the potent inhibitory effects of ritonavir both on CYP and Pgp, it is known to use ritonavir in combination with a second protease inhibitor to boost or enhance the efficacy of that second protease inhibitor. Depending on the type of second protease inhibitor employed, boosting or enhancement may be achieved primarily through the inhibition by ritonavir at the gut wall level or by hepatic metabolism.

In such combinations, ritonavir is being used to boost the effect of another agent, and not as an active agent in its own right. Accordingly, whereas ritonavir can be dosed at 600 mg BID to achieve a therapeutic effect, when used as a booster, it is used at a much reduced dose, typically 100 mg BID.

However, given the range of affinities of the protease inhibitors towards CYP and indeed even Pgp, it remains elusive, based on the prior art, to predict for a given combination of protease inhibitors, what composition or regimen will prove effective and well tolerated.

Darunavir poses a particular problem. Darunavir is a second generation protease inhibitor and the compound and its synthesis are described in U.S. Pat. No. 7,829,564. Relatively little is known regarding the interaction of this drug with ritonavir, compared that is with some of the older protease inhibitors. However, it is known to be a potent inhibitor of both CYP and Pgp. Particular compositions and regimens will be required to prevent darunavir competing with ritonavir as an inhibitor of Pgp and CYP at the gut wall, as well as an inhibitor of hepatic CYP.

There remains a need for efficacious dosage forms and regimens containing a combination of ritonavir and darunavir. More particularly, there remains a need for such dosage forms that enhance Cmax, AUC and elimination half life of darunavir. Furthermore, there is a need for such dosage forms that employ low doses of the drugs to reduce as much as possible any adverse effects or issues of tolerability. Still further, there remains a need to provide such dosage forms that can be administered in a convenient manner to patients in need of treatment and which diminish inter-patient variability in drug exposure, eliminate food requirements, reduce dosing and reduce tablet load and volumes.

The present invention addresses shortcomings in the prior art by providing dosage forms and regimens, which deliver drugs with non-linear release rates in a time-dependent manner.

The invention provides in a first aspect a dosage form suitable for once-a-day or twice-a-day administration to a patient in need of treatment comprising ritonavir or a pharmaceutically acceptable salt thereof comprising a first phase adapted for immediate release of ritonavir or a pharmaceutically acceptable salt thereof and a second phase adapted for modified release of ritonavir or a pharmaceutically acceptable salt thereof.

The term “immediate release” refers to a drug's release rate, i.e. the quantity of drug released from a dosage form per unit time, e.g. milligrams of drug released per hour (mg/hr). A drug's release rate may be measured using suitable in-vitro dissolution testing conditions known in the art. A drug release rate obtained at a particular time following administration of a dosage form refers to an in-vitro release rate obtained at the specified time using a dissolution test method.

A commonly used reference measurement for evaluating drug release rates from an oral dosage form is the time at which 90% of the drug within a dosage form has been released. This measurement is referred to as the T₉₀ for the particular dosage form tested.

In particular, the release rate of a drug from a dosage form can be determined by standard pharmacopoeia method using either a basket stirring apparatus (apparatus I) or a paddle stirring apparatus (apparatus II). Dissolution media are also described in the pharmacopoeia. In the present invention USP Apparatus II at 75 rpm may be employed and the media used may be 900 ml 60 mM polyoxyethylene 10 laurylether at 37° C.

The quantity of drug released over time may be measured by a suitable means e.g. u.v. analysis, HPLC, or other suitable analytical technique, and expressed as a percentage release (w/w) of the total weight of drug.

An immediate release dose of a drug as used in the present invention refers to a dose or phase that is substantially completely released within a time period of about 1 hour or less, and more particularly within about 30 minutes or less after the administration to a patient, or after commencement of an in-vitro dissolution test.

The term “modified release” as used hereinabove, likewise refers to a drug release rate determined after administration to a patient or after commencement of an in-vitro dissolution test. More particularly, it relates to a phase or dose that is adapted to release a drug at a certain release rate, or at a certain location (within the body) to accomplish a therapeutic objective that is not possible using an immediate release dose or phase.

More particularly, it includes delayed release, by which is meant that there can be a period of latency after administration when no drug is released followed by drug release. When drug is released, it may conform to an immediate release profile as referred to above, or it may be released at a rate that is slower and therefore sustained compared with an immediate release rate.

As well as characterising a dosage form by reference to an in-vitro measurement such as its T₉₀, it is also possible to characterise a dosage form by referring to the rate at which it releases a drug during a certain time period (“periodic release rate”). More particularly, a periodic release rate refers to the quantity of drug released from a dosage form during a specific periodic interval as determined at the end of that interval. For example, the quantity of drug released at T=1 hour is the periodic release rate of a dosage form during the first hour following administration.

In a particular embodiment of the present invention, the dosage form is adapted to release ritonavir with a descending release rate.

The term “descending release rate” refers to a release-rate profile in which the release rate over a periodic interval is lower than a preceding periodic release rate. In determining this profile, the periodic intervals may be of the same or different duration. For example, said preceding periodic release rate may be measured from T=0 through T=1 hour, and a second period may commence following T=1 hour and extend for an extended period of time, for example until T=12 hours or longer. In this case, a descending release rate is observed when the release rate in the first hour after administration is greater than the rate for the duration of the second period.

A periodic interval in respect of which a release rate is measured can commence immediately following the preceding periodic interval, or their may be a lag time between the periodic intervals. For example, ritonavir may be released immediately upon administration and for a period of about 1 hour. Thereafter, there may be a lag time between 3 to 7 hours, before a second period of release, slower than the first period, with a duration of about 2 to 5 hours.

The time period during which the descending release rate is measured may refer to a period beginning at T=0, and which continues through an extended period of time, for example, up to at least 80%, more particularly at least 90% of the drug in the dosage form is released.

In a particular embodiment the dosage form releases ritonavir in a pulsatile manner wherein the first pulse is released immediately upon administration and the second occurring at a fixed time thereafter. Suitable dosage forms that are adapted for pulsatile release are described in US20060159744, which is incorporated herein by reference.

When ritonavir is to be employed as a booster or enhancer of another co-administered protease inhibitor it is highly desirable for reasons of patient compliance and reduced incidence of adverse effects that the dose of ritonavir is as low as possible. One means of achieving greater efficacy for a given dose is to increase bioavailability of the drug, for example, by increasing its solubility in a biological medium. This can be achieved by micronisation of the drug and/or by co-formulating the drug with solubilisation aids. Alternatively or additionally, one can employ non-ionic surfactants in a dosage form containing ritonavir. Non-ionic surfactants are known to have inhibitory effects on CYP and as such, their use with ritonavir may enable reduced dosing of ritonavir.

Accordingly, in another aspect of the present invention there is provided a dosage form as hereinabove described additionally comprising a non-ionic surfactant.

A non-limiting list of non-ionic surfactants may be mentioned as strong inhibitors of CYP 3A including polysorbate 20, polyoxyl 35 castor oil, polyoxyl 40 stearate and poloxamer 188, although other non-ionic surfactants could be employed.

In another aspect of the invention there is provided a dosage regimen for once- or twice-a-day administration comprising the co-administration of darunavir, and of ritonavir in a dosage form as hereinabove described.

In yet another aspect of the present invention there is provided a dosage form suitable for once-a-day or twice-a-day administration to a patient in need of treatment comprising ritonavir or a pharmaceutically acceptable salt thereof comprising a first phase adapted for immediate release of ritonavir or a pharmaceutically acceptable salt thereof and a second phase adapted for modified release of ritonavir or a pharmaceutically acceptable salt thereof, and a third phase comprising darunavir or a pharmaceutically acceptable salt thereof.

Darunavir is a poorly soluble drug and this affects its bioavailability. Accordingly, the third phase containing darunavir may be formulated in a manner that increases its solubility in biological media and thereby improves bioavailability. The compound also exhibits a food effect, meaning that its absorption is to some extent affected by whether a patient is in a fed or fasted state when being administered the drug. Preferably dosage forms are formulated in a manner that reduces or indeed eliminates this food effect.

Various methods can be employed for increasing dissolution of poorly water-soluble ingredients. It is known that the micronization of a drug can improve its rate of dissolution, and its bioavailability. It is also known that the addition of a surfactant to a formulation of a drug is capable of improving its absorption and its bioavailability.

In one embodiment, a suspension or dispersion of darunavir may be sprayed onto an inert hydrosoluble carrier. Suspensions or dispersions may contain hydrophilic polymeric materials, surfactants or polyols.

Examples of hydrophilic polymeric materials include as polyvinylpyrrolidone, poly(vinyl alcohol), hydroxypropylcellulose, hydroxymethylcellulose, hydroxypropylmethylcellulose, gelatin, etc. Polymer blends are also suitable. Suspensions or dispersions may contain other excipients conventionally used in films and coatings.

Any surfactant is suitable, whether it is amphoteric, non-ionic, cationic or anionic. Examples of such surfactants are: sodium lauryl sulfate, monooleate, monolaurate, monopalmitate, monostearate or another ester of polyoxyethylene sorbitan, sodium dioctylsulfosuccinate (DOSS), lecithin, stearyl alcohol, cetostearylic alcohol, cholesterol, polyoxyethylene ricin oil, polyoxyethylene fatty acid glycerides, poloxamer and the like. Mixtures of surfactants are also suitable.

Examples of carriers include sugars, such as lactose, saccharose, hydrolyzed starch (maltodextrin) or mixtures thereof.

Excipients commonly used in pharmaceutical dosage forms may also be employed, such as binders, fillers, pigments, disintegrating agents, lubricants, wetting agents, buffers and the like.

The hydrosoluble carrier coated with the dispersion or suspension may be granulated and formed into a suitable dosage form for oral administration according to techniques well known in the art. Such methods and compositions are described in U.S. Pat. No. 6,277,405, which is herein incorporated by reference.

In yet another embodiment of the invention darunavir may be co-micronised with a solid surfactant.

The surfactant may be selected from solid surfactants so that it can be co-micronized with darunavir. An alkali metal sulfate of lauryl alcohol, for example sodium lauryl-sulfate may be employed, although the skilled person will appreciate that other solid surfactants could also be employed.

Co-micronization may be carried out in a milling procedure, such as air-jet milling until the powder obtained is such that the mean particle size is less than about 15 microns.

Co-micronised powder may be filled into capsules, or further formulated into other suitable oral dosage forms such as tablets or the like.

Co-micronisation methods and compositions formed therefrom are described in European patent EP 330 532, which is herein incorporated by reference.

In yet another embodiment of the present invention darunavir may be formulated with a phospholipid or an ionic surfactant such as sodium lauryl sulfate.

The use of a phospholipid or ionic surfactant can act as an aid to solubilisation of darunavir and thereby improve its bioavailability. However, it may also reduce the variability of absorption resulting from the intake by a patient of food.

Without being bound by any particular theory, it is believed that in a biological medium the phospholipid or ionic surfactant creates immediately around a drug substance an environment that mimics the environment created by the release of endogenous or natural surfactants such as phospholipids as part of the process of digestion after the intake of food. It is believed that phospholipids or ionic surfactants may interact with the drug to form micelles that in turn interact with the unstirred water layer (UWL)—a bicarbonate-rich layer of mucus that maintains pH levels at around 7 at the villi surface—and the intestinal mucosa, to enhance absorption therethrough.

The phospholipid may be a single phospholipid or a mixture of two or more phospholipids, for example a mixture of two or a mixture of three or a mixture of four or a mixture of five or a mixture of from six to about ten phospholipids. Phospholipids could be either from egg or soybean origin. Grades of phospholipids are mainly characterized by their content of phosphatidylcholine, lysophosphatidylcholine and phosphatidylethanolamine. For the present invention the preferred grade contains more than 70% of phosphatidylcholine.

Suitable phospholipids include saturated phospholipids; unsaturated phospholipids, naturally derived phospholipids, synthetic phospholipids and semi synthetic phospholipids, animal and plant phospholipids, egg phospholipids, soya bean phospholipids, corn phospholipids, wheat germ, flax, cotton, and sunflower seed phospholipids, milk fat phospholipids, purified phospholipids from these and other natural sources, glycerophospholipids, phosphatides, phospholipids containing fatty acid esters including palmitate, stearate, oleate, linoleate, and arachidonate, which esters can be mixtures and mixtures of isomers in the phospholipids, phospholipids composed of fatty acids containing one or more double bonds such as dioleoyl phosphatidylcholine and egg phosphatidylcholine that are not stable as powders but are hygroscopic and can absorb moisture and become gummy, phospholipids composed of saturated fatty acids that are stable as powders and are relatively less amenable to absorption of moisture, phosphatidyiserines, phosphatidylcholines, phosphatidylethanolamines, phosphatidylinositols, phosphatidylglycerols such as L-alpha-dimyristoyl phosphatidylglycerol also known as 1,2-dimyristoyl-sn-glycero-3-phospho (rac-1-1Jlycerol) and also known as DMPG: phosphatidic acid, hydrogenated natural phospholipids, and commercially available saturated and unsaturated phospholipids such as those available from Avanti Polar Lipids. Inc. of Alabaster, Ala., USA.

The phospholipid may be salted or desalted, hydrogenated, or partially hydrogenated. The phospholipid can be a mixture of these phospholipids.

Preferred phospholipids include Lipoid E80, Lipoid EPC, Lipoid SPC, DMPG, Phospholipon 100H, a hydrogenated soybean phosphatidylcholine, Phospholipon 90H, Lipoid SPC-3, egg phospholipid, purified egg phospholipid, and mixtures thereof. A preferred phospholipid is Lipoid E80.

The ionic surfactant may comprise one or more anionic, cationic and zwitterionic surfactant materials.

A non-exhaustive list of anionic surfactants includes ammonium lauryl sulphate, dioctyl sodium sulfosuccinate, perfluorobutanesulfonic acid, perfluorononanoic acid, perfluorooctanesulfonic acid, perfluorooctanoic acid, sodium octyl sulphate, sodium dodecylbenzenesulfonate, sodium lauroyl sarcosinate, sodium palmate, sodium stearate and triethanolamine lauryl sulphate.

A non-exhaustive list of cationic surfactants includes benzalkonium chloride, benzethonium chloride, cetrimonium bromide, cetrimonium chloride, dimethyldioctadecylammonium chloride, lauryl methyl gluceth-10 hydroxypropyl dimonium chloride, stearalkonium chloride, and tetramethylammonium hydroxide.

A non-exhaustive list of zwitterionic surfactants includes those based on primary, secondary, tertiary or quaternary ammonium surfactants.

Of course, the skilled person will appreciate that other ionic surfactants suitable for pharmaceutical use may be employed in the present invention.

The darunavir, phospholipid and/or ionic surfactant may be formulated with one or more pharmaceutically acceptable excipients.

As stated hereinabove, as a highly potent inhibitor of CYP 3A and Pgp, ritonavir dosage forms as hereinabove described may be useful boosters for darunavir, which itself is a strong substrate for both CYP 3A4 and Pgp. Furthermore, given the time dependent variable release rate of the dosage forms according to the invention there will be adequate levels of ritonavir in the gut wall at the time of dosing darunavir to ensure an elevated level of plasma Cmax for darunavir. Furthermore, to prevent hepatic metabolism of darunavir and thereby ensure elevated levels for the elimination half life of this drug, the modified release phase containing ritonavir ensures adequate plasma levels of ritonavir over time.

In a particular embodiment of the present the dosage form is formulated in a manner that ensures concomitant administration of darunavir and ritonavir. The ritonavir containing phases and the darunavir phase may be in the form of separate compositions that are to be administered at the same time, although to better ensure concomitant administration and to improve convenience of administration and therefore patient compliance it is preferred if both ritonavir and darunavir are co-formulated.

If the ritonavir and darunavir are co-formulated, the drugs may be contained in separate and discrete phases, e.g. in distinct layers of a multi-layer tablet; or one or more of the drug containing phases may be mixed together.

In yet another aspect of the present invention there is provided a method of enhancing the pharmacokinetics of darunavir or a pharmaceutically acceptable salt thereof comprising administering to a human in need of such treatment a dosage form as herein described comprising ritonavir, and a therapeutically effective amount of darunavir or a pharmaceutically acceptable salt thereof.

In still another aspect of the present invention there is provided a method of inhibiting an HIV infection comprising administering to a patient in need of such treatment dosage form as herein described comprising ritonavir, and a therapeutically effective amount of darunavir or a pharmaceutically acceptable salt thereof.

The dosage forms of the present invention may be in any form suitable for administration via the oral route. They may be in the form of capsules, tablets, pills, powders, granules, liquid formulations such as syrups, multi-particulates in powder, sachet form or suspended in a suitable liquid such as a syrup, and the like. The dosage forms may be provided in two or more discrete populations each containing a different drug or the same drug formulated in a different manner.

When the dosage forms are presented in the form of tablets, they may be formed of one or more layers. The layers may be formed concentrically or they may be formed contiguously in a sandwich-like fashion.

The dosage forms may be intended for once-a-day or twice-a-day administration.

The total daily dose of ritonavir to be administered to a human or other mammal host in single or divided doses may be in amounts, for example, from 0.001 to 300 mg/kg body weight daily and more usually 0.1 to 100 mg/kg. Dosage forms may contain such amounts of submultiples thereof to make up the daily dose.

The total daily dose of darunavir to be administered to a human or other mammal is 800 mg (given as a single or multiple dose). Dosage forms may contain such amounts of submultiples thereof to make up the daily dose.

The amount of darunavir or ritonavir that may be employed to produce a single dosage form will vary depending upon the host treated and the particular mode of administration.

It will be understood, however, that the specific dose level for any particular patient will depend upon a variety of factors including the age, body weight, general health, sex, diet, time of administration, route of administration, rate of excretion, drug combination, and the severity of the particular disease undergoing therapy.

The dosage forms described hereinabove the drugs may contain one or more pharmaceutically acceptable excipients.

Pharmaceutically acceptable excipients are employed in dosage forms of the present invention for various reasons relating to preparation and storage of the dosage form, as well as in order to achieve a desired release rate when the dosage form is finally administered in a biological medium.

These excipients typically include diluents or fillers, which add bulk to a formulation to enable formulations of a desired size to be prepared; binders or adhesives, which promote the adhesion of the particles of a formulation to maintain the integrity of a dosage form; disintegrants or disintegrating agents, which promote the break-up of the dosage form after ingestion to make the ingredients more readily available; anti-adherents, glidants or lubricants, which enhance the flow of the ingredients during handling and manufacture, for example prevention of sticking of the ingredients to tablet-making machinery; and miscellaneous adjuvants such as colourants and flavourants.

Suitable diluents include pharmaceutically acceptable inert fillers such as microcrystalline cellulose, lactose, dibasic calcium phosphate, saccharides, and/or mixtures of any of the foregoing. Examples of diluents include microcrystalline cellulose such as Avicel PH 112, Avicel PH 1O1 and Avicel PH102; lactose such as lactose monohydrate, lactose anhydrous, and Pharmatose DCL 21; dibasic calcium phosphate such as Emcompress; mannitol; starch; sorbitol; fructose; sucrose; and glucose. Diluents are selected to match the specific formulation with attention paid to the compression properties. A diluent is preferably used in an amount of 10% to 90% by weight, more particularly 50% by weight of a dosage form.

Suitable lubricants and glidants, including agents that act on the flowability of powders include colloidal silicon dioxide such as Aerosil 200, talc, stearic acid, magnesium stearate, calcium stearate, sodium stearyl fumarate, polyethylene glycol and sodium lauryl sulphate. A lubricant is preferably used in an amount of 0.5 to 2% by weight, in particular 1% by weight, of a dosage form.

Suitable binders include polyethylene glycols such as PEG 6000; cetostearyl alcohol; cetyl alcohol; polyoxyethylene alkyl ethers; polyoxyethylene castor oil derivatives; polyoxyethylene sorbitan fatty acid esters; polyoxyethylene stearates; poloxamers; waxes, alginic acids and salts thereof; HPC—hydroxypropyl cellulose; HPMC—hypromelose; methylcellulose; maltodextrin and dextrin; povidone; gums; starch and modified starches. Binders may be used in an amount of 2 to 10% by weight, more particularly 5% by weight, of a dosage form.

Suitable disintegrants include sodium starch glycolate, such as Explotab®, crospovidone such as Kollidon CL, polyplasdone XL, sodium carboxymethylcellulose, sodium croscarmellose such as AcDiSoI, and starch. Disintegrants may be used in an amount of 2 to 10% by weight, more particularly 5% by weight, of a dosage form.

When a phase of a dosage form should be adapted for modified release, it may contain any of the aforementioned adjuvants as well as one or more release-controlling agents.

The term “release-controlling agent” includes any agent that controls the release of drug either temporally or by location in order to give a therapeutic effect not possible with a conventional immediate release formulation, and includes hydrophilic polymers, hydrophobic polymers or mixtures thereof, or copolymers thereof, or mixtures of these polymers and copolymers.

Examples of release-controlling agents include hydroxyalkylcellulose, such as hydroxypropylcellulose and hydroxypropylmethylcellulose; poly(ethylene)oxide; alkylcellulose such as ethycellulose and methylcellulose; carboxymethylcellulose; hydrophilic cellulose derivatives; polyethylene glycol; cellulose acetate; cellulose acetate butyrate; cellulose acetate phthalate; cellulose acetate trimellitate; polyvinylacetate phthalate; hydroxypropylmethylcellulose phthalate; hydroxypropylmethylcellulose acetate succinate; poly(alkyl methacrylate); and poly(vinyl acetate). Other suitable hydrophobic polymers include polymers or copolymers derived from acrylic or methacrylic acid esters, copolymers of acrylic and methacrylic acid esters, zein, natural or synthetic waxes, shellac, hydrogenated vegetable oils, semi synthetic glycerides, a poly(ethylene oxide), ethylcellulose or a combination thereof. Release-controlling agents may be present in amounts of 10 to 90% based on the weight of a dosage form.

Dosage forms of the present invention may be coated with coating materials to achieve all manner of desired effects: For example coatings may be provided to achieve a release-controlling effect, an aesthetic effect (e.g. an attractive colour or pleasant taste) or information effect, e.g. a coating may be coloured to act as a visual cue for a patient identification of the correct medicament. Coatings may also be over-written with information relating to the dosage, or they may elicit a functional effect such as a handling effect, e.g. a smooth coating for ease of swallowing, or a stability effect, e.g. a moisture or light barrier during storage.

A preferred dosage form for drug combination delivery according to the invention is a multilayer tablet in which one layer could be dedicated to the sustained release fraction.

In order to facilitate the preparation of dosage forms described above there is provided, in a further aspect of the present invention, a process for the preparation of a dosage form according to the present invention. Darunavir fraction is manufactured by a dry granulation process after dry blending of the different components of the formulation, the homogeneous blend is processed between the rolls of a roller compactor.

With regard to ritonavir process of preparation is depending on expected release profile, The invention will now be further illustrated with reference to the following Examples.

### EXAMPLE 1

A coated multilayer tablet consisting of:

### 1a Darunavir Layer:

| | |
|---|---|
| Darunavir ethanolate | 433 mg |
| Prosolv SMCC (silicified MCC) | 300 mg |
| HPC | 60 mg |
| AcDiSol | 100 mg |
| Magnesium stearate | 10 mg |
| Total layer weight | 903 mg |

Darunavir ethanolate, Prosolv, HPC and 70% of AcDiSol are mixed together in a bin blender—Then the blend is passed through a Fitzpatrick chilsonator to produce ribbons that are subsequently ground in an oscillatory mill. The obtained size particles are then blended with the rest of AcDiSol and magnesium stearate in a bin blender to get a ready to tablet blend.

### 1b Ritonavir CR Layer:

| | |
|---|---|
| Ritonavir | 25 mg |
| Lipoid S 75 | 25 mg |
| Lactose | 150 mg |
| Maltodextrin | 25 mg |
| HPMC type 2910 | 65 mg |
| Sodium stearyl fumarate | 10 mg |

In a stainless steel vessel Lipoid S75 is dispersed in hot water (temp about 90° C.), then Ritonavir is added under stirring. The dispersion is then pushed trough a high pressure homogenizer to form phospholipid-coated microparticles. Then the suspension is cooled down to room temperature.

After addition of maltodextrin, the microparticle suspension is then dried by top spraying it on lactose in a fluid bed dryer. The dry material is then sized in a cone mill. The powder is then mixed with HPMC and sodium stearyl fumarate in a bin blender

### 1c Tabletting

On a multilayer tablet press, two-layer tablets with a total weight of 1203 mg are prepared with oblong shape tooling size 19×9 mm. The first layer (pressed with 2 stations) is adjusted at 903 mg weight whereas the second layer is set at 300 mg

### 1d Ritonavir Immediate Release Fraction.

| | |
|---|---|
| Ritonavir | 25 mg |
| Lipoid S 75 | 25 mg |
| Poloxamer 188 | 10 mg |
| Sucrose | 150 mg |

In a stainless steel vessel Lipoid S75 is dispersed in hot water (temp about 90° C.), then Ritonavir is added under stirring. The dispersion is then pushed trough a high pressure homogenizer to form phospholipid coated microparticles. After the suspension has been cooled down sucrose is added under stirring. The obtained suspension is then applied in a pan coater by spraying onto the tablets manufactured in 1c for a weight increase of 210 mg/tablet (16%) corresponding to 25 mg of ritonavir.

### EXAMPLE 2

A multilayer tablet consisting of 1a, 1b and an immediate release layer prepared according to the following process. (2c)

| | |
|---|---|
| Ritonavir | 25 mg |
| Lipoid S 75 | 25 mg |
| SLS | 15 mg |
| Lactose | 150 mg |
| Maltodextrin | 25 mg |
| AcDiSol | 50 mg |
| Sodium stearyl fumarate | 10 mg |

In a stainless steel vessel Lipoid S75 is dispersed in hot water (temp about 90° C.), then Ritonavir is added under stirring. The dispersion is then pushed trough a high pressure homogenizer to form phospholipid-coated microparticles. Then the suspension is cooled down to room temperature.

After addition of maltodextrin and SLS (50%), Microparticle suspension is then dried by top spraying it on lactose in a fluid bed dryer. The dry material is then sized in a cone mill. The powder is then mixed with AcDiSol, SLS (rest) and sodium stearyl fumarate in a bin blender.

### 2d Tabletting Step.

On a multilayer tablet press a multilayer tablet is prepared with a first layer corresponding to example 1a, a second layer corresponding to example 1b and a third layer corresponding to example 2c for a final tablet weight of 1503 mg and a final tablet size of 21×10 mm. This tablet is subsequently film coated in a pan coater for light and moisture protection.

1. A dosage form suitable for once-a-day or twice-a-day administration to a patient in need of treatment comprising a first phase adapted for immediate release and containing ritonavir or a pharmaceutically acceptable salt thereof; and a second phase adapted for modified release and containing ritonavir or a pharmaceutically acceptable salt thereof.

2. A dosage form according to claim 1 adapted to release ritonavir or a pharmaceutically acceptable salt thereof with a descending release rate.

3. A dosage form according to claim 2 wherein the release rate during a second period is lower than the release rate during a preceding period.

4. A dosage form according to claim 3 wherein the immediate preceding period commences at T=0 until T=1 hour, and the second period extends up to T=12 hours.

5. A dosage form according to claim 1 wherein ritonavir is released in pulses; the first being immediate upon administration and having a duration of about 1 hour; the second being delayed between 3 to 7 hours and having a duration of 2 to 5 hours.

6. A dosage form according to claim 1 comprising a non-ionic surfactant that is an inhibitor of CYP 3A.

7. A dosage form according to claim 6 wherein the surfactant is selected from the group consisting of polysorbate 20, polyoxyl 35 castor oil, polyoxyl 40 stearate and poloxamer 188.

8. A dosage form according to claim 1 comprising darunavir.

9. A dosage form according to claim 8 wherein the darunavir is co-formulated with ritonavir.

10. A dosage form according to claim 8 wherein darunavir is in a separate and discrete phase from ritonavir.

11. A method of enhancing the pharmacokinetics of darunavir or a pharmaceutically acceptable salt thereof comprising administering to a human in need of such treatment a therapeutically effective amount of darunavir or a pharmaceutically acceptable salt thereof and a dosage form as defined in claim 1.

12. A method of inhibiting an HIV infection comprising administering to a patient in need of such treatment a therapeutically effective amount of darunavir or a pharmaceutically acceptable salt thereof and a dosage form a defined in claim 1.

13. A dosage form according to claim 1, wherein the total daily dose of ritonavir is from 0.001 to 300 mg/kg body weight.

14. A dosage form according to claim 13, wherein the total daily dose of ritonavir is from 0.1 to 100 mg/kg body weight.

15. A dosage form according to claim 1, wherein the dosage form comprises 50 mg ritonavir.

16. A dosage form according to claim 1, wherein the amount of ritonavir in the first phase is equal to the amount of ritonavir in the second phase.

17. A dosage form according to claim 1 comprising a second protease inhibitor.

18. A dosage form according to claim 12, wherein darunavir is co-micronised with a solid surfactant.

19. A dosage form according to claim 12, wherein darunavir is formulated with a phospholipid or an ionic surfactant or both.

20. The method of claim 12, wherein the dosage form comprises a sub-therapeutic dose of ritonavir.
